SEQUENCE LISTING (6 sequences)

SEQ ID NO: 1 - DNA, 23 bp, Artificial Sequence
  Synthesized
gcagtccgttgaggaaacttacg

SEQ ID NO: 2 - DNA, 23 bp, Artificial Sequence
  Synthesized
catacactggattagaggcatgc

SEQ ID NO: 3 - DNA, 22 bp, Artificial Sequence
  Synthesized
gtgactatgtcgaagcgaccag

SEQ ID NO: 4 - DNA, 22 bp, Artificial Sequence
  Synthesized
gcctgttccttcattccagagg

SEQ ID NO: 5 - DNA, 21 bp, Artificial Sequence
  Synthesized
tcacggttgcggtactgggct

SEQ ID NO: 6 - DNA, 21 bp, Artificial Sequence
  Synthesized
ccacgcccgtctcgaaggttc

(19) United States
(12) Patent Application Publication
(10) Pub. No.: US 2015/0040268 A1
(43) Pub. Date: Feb. 5, 2015

(54) METHODS AND COMPOSITIONS FOR THE DELIVERY OF NUCLEIC ACIDS TO SEEDS

(71) Applicant: MORFLORA ISRAEL LTD, MOSHAV SHARSHERET (IL)

(72) Inventors: MIRI MIRIAM LAPIDOT, MEITAR (IL); IRIS SMIRRA, BITSARON (IL); HERVE HUET, YEHUD (IL); DOTAN PELEG, KIRYAT TIVON (IL); OMRI LEMPEL, BAT-YAM (IL)

(73) Assignee: MORFLORA ISRAEL LTD, MOSHAV SHARSHERET (IL)

(21) Appl. No.: 14/260,952

(22) Filed: Apr. 24, 2014

Related U.S. Application Data
(60) Provisional application No. 61/816,044, filed on Apr. 25, 2013.

(51) Int. Cl. C12N 15/82

(52) U.S. Cl. CPC C12N 15/8283
USPC 800/279; 800/301

(57) ABSTRACT

The present invention relates to methods of seed treatment and introduction of nucleic acid particles into intact seeds. In particular, the methods are non-priming seed treatment protocols capable of delivering naked DNA plasmids into seeds, without the use of microorganism or any additional means, and which are not plant species limited.

### CROSS REFERENCE TO RELATED APPLICATIONS

This application claims the benefit of priority from U.S. provisional patent application Ser. No. 61/816,044 filed on Apr. 25, 2013, which is incorporated herein by reference in its entirety.

### BACKGROUND OF THE INVENTION

The present invention relates to methods of seed treatment and introduction of nucleic acid particles into intact seeds. In particular, the methods of the present invention are non-priming seed treatment methods capable of delivering nucleic acid into seeds, without the use of microorganisms or any additional means, and which are not plant species limited. All publications cited in this application are herein incorporated by reference.

Plants carrying one or more expressible heterologous genes have a variety of potential advantages. The plants carrying a gene expression cassette may carry one or more genes which confer desired traits, including for example, herbicide, pesticide or insect tolerance; tolerance to stress; enhanced flavor and/or shelf life of the fresh produce (fruit, seeds etc.), as well as the ability to synthesize useful plant and foreign proteins, sugars, fatty-acids or secondary metabolites for consumption by man and/or animals or for use as raw materials in a variety of industries (cosmetics, pharmaceuticals, nutraceutics, foods, paper, fibers, etc.).

Current transformation technologies provide an opportunity to engineer plants with desired traits, and major advances in plant transformation have occurred in recent years. The most common technology is mediated by Agrobacterium tumefaciens. Other methods of transformation are direct DNA transfer including microinjection, electroporation, and particles bombardment and viral vectors (Birch R G. 1997. Annu Rev. Plant Physiol. Plant Mol. Biol. 48:297-326). With the exception of viral vectors, application of the above techniques results in the penetration of foreign DNA into the treated cells/tissue, integration of the foreign DNA into the treated plant's genome, expression and following selection, the regeneration of transgenic plants. However, in many major crop plants, serious genotype limitations still exist.

Transformation of some agriculturally important crop plants (e.g., sweet pepper) continues to be both difficult and time consuming. In addition, in all the aforementioned technologies, transformation is conducted with vegetative tissues (including embryos) and the efficiency of transformation is poor, markers for selection are required, and the percentage of successful regeneration of a plant from the transformed cell or tissue is rather low.

Integration of foreign DNA into a plant genome is not always desirable, particularly when genetically modified (GMO) plants raise environmental and political issues. In these cases, methods enabling the expression of the heterologous gene(s) while the genes are not incorporated into the host genome are desired.

For this effect, it has been demonstrated that plant viruses can be used as gene expression and silencing vectors. Foreign sequences have been introduced into the genomes of plant viruses (and not in the plant genome) in such a way that their ability to replicate is retained. As a result, the foreign sequence is amplified resulting in either the expression of a foreign protein or virus-induced gene silencing of an endogenous plant gene. An important and limiting step for the use of viral vectors is infection of the plants with the viral replicons. Full-length complementary DNA (cDNA) clones of many RNA plant viruses have been produced and shown to be infectious either after transcription in vitro with the appropriate RNA polymerase or when directly applied as DNA under the control of a plant-specific promoter to drive in vivo transcription.

Use of Agrobacterium to deliver a copy of both DNA and RNA virus-based vectors encoded on the T-DNA (‘agrodelivery’) provides an excellent solution since Agrobacterium is an extremely efficient vector. All the steps that are necessary for the conversion of the ‘agrodelivered’ T-DNA into a functional DNA or RNA replicon have been shown to occur in plants. ‘Agroinfection’ has been used for many years. It is also often much more efficient than mechanical inoculation using viral particles, and is definitely more efficient than using DNA or RNA as infectious molecules. (Lomonossoff and Montague, 2008; Gleba, 2008) However, virus vector delivery, either mechanically, biolistically or by Agrobacterium cannot be applied on a very large scale.

Therefore, developing novel, fast and simple methods for delivering DNA to commodity seed crops is highly desirable.

### SUMMARY OF THE INVENTION

The present invention provides for the introduction of nucleic acid particles, such as dsDNA or ssDNA, particularly heterologous DNA, via simple and efficient non-priming seed treatment methods, using particularly modified commercial non-priming seed treatment protocols without the use of microorganisms or any additional means, and which is not plant species limited. Surprisingly, the non-priming seed treatment methods of the present invention result in uptake of heterologous DNA in the germinated plants.

In one aspect, the present invention provides means and methods for introduction of nucleic acids into plants using a non-priming seed treatment. Particularly, the invention combines modified commercial seed treatments (such as coating, film coating, dressing and pelleting) and the uploading of heterologous DNA into a plant seed. The introduced heterologous DNA is unexpectedly able to enter and be incorporated into the seed embryo cells during germination. Particularly, one aspect of the invention provides means and methods of uploading viral-based DNA plasmids, which can systemically replicate and spread in the germinated plants without integrating to the plant genome. In another aspect, although carrying viral elements, there is no release of virus particles into the plant tissues by the viral-based DNA plasmid.

In another aspect, the present invention provides means and methods of uploading naked DNA plasmids into seeds using non-priming seed treatment, without the use of microorganisms or any additional means, and which is not plant species limited. Particularly, the invention combines modified commercial seed treatments (such as coating, film coating, dressing and pelleting) and the uploading of heterologous DNA into a plant seed. The coating for a seed may comprise any commercial binder, that may comprise one or more binders selected from the group consisting of polymers and copolymers of polyvinyl acetate, methyl cellulose, polyvinyl alcohol, vinylidene chloride, acrylic, cellulose, polyvinylpyrrolidone and polysaccharide and the a naked DNA wherein the binder forms a matrix for the heterologous DNA. The applied heterologous DNA is unexpectedly able to reach the seed embryo cells during germination.

In another aspect, the present invention is advantageous over previously know methods for plant cell transformation or introduction of foreign DNA into plants, in that the nucleic acid particles are introduced into intact crop seeds which are naturally capable of developing into a whole plant, without the need for regeneration cultures. Surprisingly, the process thus does not include the use of microorganisms, nor virus particles or any addition of other biologic materials.

In another aspect, the present invention for DNA introduction does not involve damage to the cells or the whole seed. In addition, the seed treatment methods of the present invention are simple and efficient, with no impact on seed germination. The methods can be large-scaled to reach commercial applications for commodities.

In a further aspect, the non-priming seed treatments of the present invention are very quick and short in time (ranging anywhere between approximately 1 second to approximately 60 minutes, including any integer or fraction thereof), and the coating/dressing/pelleting is external to the seed and does not trigger physiological changes. The uploading of the DNA is on the surface of the seed coat. Conversely, the priming seed treatment is a much longer process (from approximately 12 hours to 72 hours or longer) and during priming there is initiation of physiological processes inside the seeds, such as enzymatic activity. In non-priming treatments of the present invention, the nucleic acid is expected to enter into the embryo cells only after sowing the seed in the field and irrigating, whereas in priming treatments, the nucleic acid will enter into the embryo cells during the priming process itself.

In a further aspect of the present invention, the introduced heterologous DNA can be of a non-integrating mode of function such as in the case of using a plant virus vector, which is not incorporated into the plant genome, but is still capable of replicating and spreading in the germinating seed-treated plant for its whole life span. A foreign gene can be introduced in the virus vector, allowing its transient expression.

In a further aspect of the present invention, the introduced heterologous DNA can be geminivirus-based expression constructs, the heterologous DNA is not incorporated into the plant genome, but is still capable of replicating and spreading in the germinating seed-treated plant for its whole life span, without passing to the next generation. The heterologous DNA may be present as naked DNA in a suitable expression vector such as plasmid, or in a virus vector-based DNA construct which is preferable in accordance with the invention.

According to certain aspects, the virus-based DNA construct comprises viral genes or parts thereof enabling replication and symptomless spreading of the construct into adjacent plant cells.

In a further aspect of the present invention, the DNA construct is a Geminivirus based construct. According to these embodiments, the construct comprises the heterologous DNA flanked by a non-contiguous nucleic acid sequence encoding Geminivirus replicase or replicase-associated protein.

According to other aspects of the present invention, the construct further comprises a polynucleotide sequence encoding a modified Geminivirus coat protein (CP). According to typical embodiments, the modified Geminivirus coat protein encoding polynucleotide comprises a mutation or deletion in nucleotides encoding the N-terminal 100 amino acids.

According to further typical aspects of the present invention the expression construct further comprises a polynucleotide sequence encoding a modified Geminivirus V2 protein.

According to additional typical aspects of the present invention the expression construct further comprises a polynucleotide sequence encoding a modified Geminivirus C4 protein. According to these embodiments, the modified Geminivirus C4 protein includes a mutation or deletion. According to certain embodiments the expression construct further comprises a bacterial polynucleotide sequence.

According to additional typical aspects of the present invention, the expression construct is devoid of a polynucleotide sequence encoding the Geminivirus C2 and/or C3 coding sequence.

According to certain currently preferred aspects of the present invention, the Geminivirus is Tomato Yellow Leaf Curl Virus (TYLCV). According to these embodiments, the Geminivirus-based construct is selected from the group denominated TraitUP, comprising IL-60, p1470 and their derivatives including their transactivatable and inducible pIR satellite-like constructs.

According to certain aspects of the present invention, the DNA construct is designed as an expression construct such that the heterologous DNA is expressed in the plant cell. According to these embodiments, the DNA construct further comprises at least one regulatory element selected from the group consisting of an enhancer, a promoter, and a transcription termination sequence.

According to additional aspects of the present invention, the DNA construct further comprises a marker for identifying the seeds and/or plants comprising the heterologous DNA.

In an aspect of the present invention, the heterologous DNA can encode any desired product, including peptides, polypeptides, proteins and RNAs. The proteins or RNAs can be of plant origin or of other origin, including bacterial and mammal origin. According to certain embodiments, the DNA encodes an inhibitory RNA selected from the group consisting of antisense mRNA, dsRNA, siRNA and the like, such that the expression of a target gene is silenced. According to other embodiments, the heterologous DNA encodes a product the expression of which confers a desirable agronomic trait including, but not limited to, resistance to biotic or abiotic stress, increased yield, increased yield quality, preferred growth pattern and the like. According to additional embodiments, the encoded protein products are useful in the cosmetic or pharmaceutical industry. According to still further embodiments, the encoded protein enhances the production of desired metabolites within the plant cells.

According to yet other aspects of the present invention, the heterologous DNA does not encode a desired product but is present merely as a label to the origin of the seed, or to the plant grown from said seed, for example to prevent illegal distribution of proprietary seeds, plants and tissues.

In another aspect of the present invention, the heterologous DNA may be transiently expressed in the cells and cells derived therefrom or it may be incorporated into the cell genome. The heterologous DNA may be present in the cytoplasm of the plant cell, in its organelles or in the nucleolus as an integrated or non-integrated DNA sequence.

According to certain currently preferred aspects of the present invention the DNA construct is a Geminivirus-based construct designed such that the heterologous DNA is not incorporated into the cell genome but the construct is capable of replicating and spreading within the cells of the plant grown from the seed comprising said heterologous DNA.

In another aspect, the methods of the present invention can be employed with seeds of any plant of interest, including geminivirus host and non-host plants, and they are fast and efficient methods, which can be applied particularly for commodity seeds.

According to yet additional aspects, the present invention provides a seed produced by the methods of the invention, the seed comprising a heterologous DNA (either naked, or in a suitable construct and preferably in a virus-based construct), and plants or parts thereof produced from said seed.

According to certain aspects, the plant seed is of a monocot origin. According to other embodiments, the plant seed is of a dicot origin.

Other objects, features and advantages of the present invention will become clear from the following description and drawings.

### SUMMARY OF THE SEQUENCE LISTING

The present application is being filed along with a Sequence Listing in electronic format. The information in the electronic format of the Sequence Listing is part of the present application and is incorporated herein by reference in its entirety.

SEQ ID NO:1 sets forth the sequence of Primer A.

SEQ ID NO:2 sets forth the sequence of Primer B.

SEQ ID NO:3 sets forth the sequence of Primer C.

SEQ ID NO:4 sets forth the sequence of Primer D.

SEQ ID NO:5 sets forth the sequence of Primer E.

SEQ ID NO:6 sets forth the sequence of Primer F.

### BRIEF DESCRIPTION OF THE DRAWINGS

FIG. 1. (A) Shows soybean seeds before and after seed treatment by the methods of the present invention. On the left side are shown soybean seeds before treatment and on the right side are shown soybean seeds after treatment with addition of 1.5% polyethylene glycol (PEG). (B) Shows soybean seeds that have been treated by the methods of the present invention during germination process. The cotyledons are still partially covered by the red coat.

FIG. 2. Shows a schematic structure of the TraitUP plasmid No. p1470. The plasmid is a pDrive bacterial vector containing a viral based fragment that includes several TYLCV genes (or part of) to be theoretically expressed as follows: gene CP encoding for the coat protein of the virus (CP=coat protein=V1); with a 20 amino-acid deletion in its N-terminal; gene V2 (encoding for the so called pre-coat), also with 20 amino-acid deletion; and truncated gene C1/C4, encoding for the N terminal of C1—the Replication initiator protein (284 bp out 1071 bp) and N terminal of gene C4 (133 bp out 291 bp). In addition, the plasmid vector comprises the IR region: IR, represent the inter region of TYLCV, sized of about 313 nucleotides. This fragment sequence contains the promoter sequence for transcription of the viral-sense ORF (V2 and V1) and the complementary sense partial ORF C1 and C4 and it is crucial for the viral DNA replication.

FIG. 3. Shows PCR analysis to verify the presence of p1470 in seedlings of soybean seeds treated by the methods of the present invention. PCR analysis was performed on 14 day old seedlings of soybean that were seed-treated as described in Example 2. DNA was extracted from true leaves and PCR amplification was performed with primers for the p1470 plasmid component (PCRI with primers A and B, on the coat protein gene, followed by PCRII with primers C and D, on the coat protein gene, expected fragment size 543 bp). Lane 1 shows the PCR mix of PCRI with no DNA, lane 2 shows the PCR mix of PCRII with no DNA, lanes 3-4 show control untreated plant, lane 5 shows the 1 Kb DNA ladder RTU (Ready-to-Use) (Genedirex), lanes 6-19 show treated plants, and lane 20 shows the p1470 plasmid.

FIG. 4. Shows a Table summarizing the success of delivery of dsDNA plasmid using various coating mediums on soybean seeds treated by the methods of the present invention.

FIG. 5. Shows PCR analysis to verify the presence of p1470 in seedlings of corn seeds treated by the methods of the present invention. PCR analysis was performed on 12 day old seedlings of corn that were seed-treated as described in Example 4. DNA was extracted from true leaves and PCR amplification was performed with primers for the p1470 plasmid component (E-F, on the coat protein gene, expected fragment size 420 bp). Lane 1 shows the PCR mix with no DNA, lane 2 shows the 1 Kb DNA ladder RTU (Ready-to-Use) (Genedirex), lanes 3-5 show control untreated plants, lanes 6-11 show treated plants, and lane 12 shows the p1470 plasmid.

FIG. 6 Shows PCR analysis to verify the presence of p1470 in seedlings of canola seeds treated by the methods of the present invention. PCR analysis was performed on 5 week old seedlings of canola that were seed treated as described in Example 5 and sown six months after coating. DNA was extracted from true leaves and PCR amplification was performed with primers for the p1470 plasmid component (C-D, on the coat protein gene, expected fragment size 543 bp). Lane 1 shows the PCR mix with no DNA, lane 2 shows the 1 Kb DNA ladder RTU (Ready-to-Use) (Genedirex), lanes 3-5 show control untreated plants, lanes 6-12 show treated plants, and lane 13 shows the p1470 plasmid.

FIG. 7 Shows PCR analysis to verify the presence of p1470 in seedlings of rice seeds treated by the methods of the present invention. PCR analysis was performed on one month old seedlings of rice that were seed treated as described in Example 6. DNA was extracted from roots and PCR amplification was performed with primers for the p1470 plasmid component (C-D, on the coat protein gene, expected fragment size 543 bp). Lane 1 shows the PCR mix with no DNA, lane 2 shows the 1 Kb DNA ladder RTU (Ready-to-Use) (Genedirex), lanes 3-6 show control untreated plants, lane 7 is blank, lanes 8-17 show treated plants, and lane 18 shows the p1470 plasmid.

FIG. 8 Shows nested PCR analysis to verify the presence of p1470 in seedlings of melon seeds treated by the methods of the present invention. PCR analysis was performed on 19 day old seedlings of melon that were seed treated as described in Example 8. DNA was extracted from true leaves and PCR amplification was performed with primers for the p1470 plasmid component (A-B, on the coat protein gene). Lane 1 shows the PCR mix of PCRI with no DNA, lane 2 shows the PCR mix of PCRII with no DNA, lanes 3-4 show control untreated plant, lane 5 shows the 1 Kb DNA ladder RTU (Ready-to-Use) (Genedirex), lanes 6-21 show treated plants, and lane 22 shows the p1470 plasmid.

### DETAILED DESCRIPTION OF THE INVENTION

In the description and tables that follow, a number of terms are used. In order to provide a clear and consistent understanding of the specification and claims, including the scope to be given such terms, the following definitions are provided:

3′ non-coding sequences. The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht I L. et al. (1989. Plant Cell 1:671680).

Allele. The allele is any of one or more alternative form of a gene, all of which relate to one trait or characteristic. In a diploid cell or organism, the two alleles of a given gene occupy corresponding loci on a pair of homologous chromosomes.

Cell. Cell as used herein includes a plant cell, whether isolated, in tissue culture or incorporated in a plant or plant part.

Coating color. As used herein, refers to coloration added to a non-priming seed treatment medium used to visually verify even coverage of the seed treatment on the seeds. Also referred to as ‘bio color’.

Commercial seed treatments. Include but are not limited to seed treatments such as coating, film coating, dressing and pelleting.

Commodity seeds/Commodity crops. Commodity seeds or crops are any seeds or crops that are traded and used commercially. Generally, they are relatively nonperishable, storable, transportable, and undifferentiated. Commodity crops are crops grown, typically in large volume and at high intensity, specifically for the purpose of sale to the commodities market, as opposed to direct consumption or processing. Some examples include, but are not limited to, corn, soybean, wheat, cotton, rice and the like.

Comprising the heterologous DNA. The term “comprising the heterologous DNA” when used in reference to a plant or seed refers to a plant or seed that contains at least one heterologous DNA in one or more of its cells. As used herein, the term refers to a plant, a plant structure, a plant tissue, a plant seed or a plant cell that contains at least one heterologous DNA in at least one of its cells. This term includes the primary cell to which the DNA was introduced and cultures and plants derived from that cell without regard to the number of transfers. All progeny may not be precisely identical in DNA content, due to deliberate or inadvertent mutations. Mutant progeny that have the same functionality as screened for in the original cell to which the DNA was introduced are included in the term.

Construct. The term “construct” as used herein, refers to an artificially assembled or isolated nucleic acid molecule which includes the heterologous DNA interest. In general a construct may include the heterologous DNA, typically a gene of interest, a marker gene which in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

Cotyledon. A cotyledon is a type of seed leaf. The cotyledon contains the food storage tissues of the seed.

Diploid. A cell or organism having two sets of chromosomes.

Embryo. The embryo is the small plant contained within a mature seed.

Enhancer. As used herein, the term an “enhancer” refers to a DNA sequence which can stimulate promoter activity, and may be an innate element of the promoter or a heterologous element inserted to enhance the level or tissue-specificity of a promoter.

Expression. As used herein, refers to the production of a functional end-product e.g., an mRNA or a protein

Gene. The term “gene” refers to a nucleic acid (e.g., DNA or RNA) sequence that comprises coding sequences necessary for the production of RNA or a polypeptide. The term comprises natural as well as man tailored (synthetic) genes. A polypeptide can be encoded by a full-length coding sequence or by any part thereof. The term “parts thereof” when used in reference to a gene refers to fragments of that gene ranging in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either. The sequences which are located 5′ of the coding region and which are present on the mRNA are referred to as 5′ non-translated (or untranslated) sequences (5′ UTR). The sequences which are located 3′ or downstream of the coding region and which are present on the mRNA are referred to as 3′ non-translated (or untranslated) sequences (3′ UTR).

Gene Silencing. The interruption or suppression of the expression of a gene at the level of transcription or translation.

Genotype. Refers to the genetic constitution of a cell or organism.

Haploid. A cell or organism having one set of the two sets of chromosomes in a diploid.

Heterologous DNA. The terms “heterologous DNA” or “exogenous DNA” refer to a polynucleotide that is not present in its natural environment (i.e., has been altered by the hand of man). For example, a heterologous DNA includes a polynucleotide from one species introduced into another species. As used herein, a heterologous DNA also includes a polynucleotide native to an organism, which may or may not have been altered in some way (e. g., mutated, added in multiple copies, linked to a non-native promoter or enhancer sequence, etc.). Heterologous DNA may comprise gene sequences of plant, bacteria and mammal origin. The gene sequences may comprise cDNA forms of a gene; the cDNA sequences may be expressed in either a sense (to produce mRNA) or anti-sense orientation (to produce an anti-sense RNA transcript that is complementary to the mRNA transcript). Heterologous plant genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

Locus. A defined segment of DNA.

Naked DNA. Refers to DNA that is devoid of protein coat, or included into a microorganism. For the transfer of genes, naked DNA usually consists of a bacterial plasmid vector with or without plant viral elements and containing the sequence or the gene to be transferred. As used herein, “naked DNA” can be synthetic or purified from a microorganism, and can also be associated with some chemicals to help its penetration into a germinating embryo.

Non-priming medium. Refers to the mediums used in the non-priming seed coating, film coating, seed dressing and seed pelleting seed treatments of the present invention.

Non-priming seed treatment. Non priming seed treatments include but are not limited to, seed dressing, seed coating and film coating, and seed pelleting. In addition to heterologous DNA, chemical agents used in agriculture, such as herbicide, pesticides, growth hormones, etc., can be added in these treatments, which are up-taken by the plant emerging from the seeds, increasing protection or growth vigor. Non-priming seed treatments of the present invention add between approximately 0.5% to 5% w/w hydration to the seeds; however, as used herein, non-priming seed treatments also include hydration up to 20% w/w. For example, 0.5%, 1%, 1.5%, 2.3%, 3.7%, 4.5%, 6.7%, 9.5%, 11.4%, 13.6%, 15.9%, 17.2%, 18.8%, 19.4%, or 20% w/w including any integer or fraction thereof, including any hydration added between 0.5% and 20% w/w may be added to the seeds during the non-priming treatment method of the present invention. Conversely, priming seed treatments increase seed water content by approximately 25% to 45% w/w. Non-priming seed treatments are very quick and short in time (from approximately 1 second to approximately 60 minutes, for example, 1.0 second, 2.3 seconds, 3.1 seconds, 6.2 seconds, 11.7 seconds, 59.0 seconds, 84.5 seconds, 118.3 seconds, 169.4 seconds, 214.1 seconds, 240.0 seconds, 5 minutes, 12 minutes, 28 minutes, 46 minutes, or 60 minutes including any integer or fraction thereof), and the coating/dressing/pelleting is external to the seed and does not trigger physiological changes. Conversely, priming seed treatment is a much longer process (from approximately 12 hours to 72 hours or longer) and during priming there is initiation of physiological processes inside the seeds, such as enzymatic activity. In non-priming treatments of the present invention, a plasmid will enter into the embryo cells only after sowing the seed in the field and irrigating, whereas in priming treatments, a plasmid will enter into the embryo cells during the priming process itself.

Nucleic Acid. The term “nucleic acid” as used herein refers to DNA that is linear or branched or circular, single or double stranded, or a hybrid thereof.

Operably linked. The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

Pathogen. Any of a number of microorganisms, such bacteria, fungi and viruses that can impart a disease to a plant or seed.

Plant. As used herein, the term “plant” includes reference to an immature or mature whole plant, including a plant from which seed, grain, or anthers have been removed. Seed or embryo that will produce the plant is also considered to be the plant.

Plant parts. As used herein, the term “plant parts” (or a plant organ, or a part thereof) includes but is not limited to protoplasts, leaves, stems, roots, root tips, anthers, pistils, seed, grain, embryo, pollen, ovules, cotyledon, hypocotyl, pod, flower, shoot, tissue, petiole, cells, meristematic cells, and the like.

Plant tissue. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be within the plant, in organ culture, tissue culture, or cell culture.

Progeny. As used herein, includes an F₁ plant produced from the cross of two plants where at least one plant includes, but is not limited to, subsequent F₂, F₃, F₄, F₅, F₆, F₇, F₈, F₉, and F₁₀ generational crosses with the recurrent parental line.

Promoter. The terms “promoter element,” “promoter,” or “promoter sequence” as used herein, refer to a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. New promoters of various types useful in plant cells are constantly being discovered; numerous examples may be found in Okamuro J K and Goldberg R B (1989) Biochemistry of Plants 15:1-82.

Seed coating. As used herein, refers to the external coverage of a seed with a medium containing a binder that is diluted with a small amount of water and, in the present invention, heterologous DNA in the form of naked or virus-based DNA plasmid. Chemical agents, used in agriculture, such as herbicide, pesticides, fungicides, growth hormones, etc., can be added in the seed coating, which are up-taken by the plant emerging from the seeds, increasing protection or growth vigor. The coating medium may be composed of a binder diluted in water (binder can be 30-100% of the medium), the introduced plasmid (one or more) (in a certain dosage range from 0.1 to 10 μg DNA per seed), coating color or bio color (0% to 30%), with or without the addition of additives such as, but not limited to, polysaccharides (for example, PEG), abrasive materials (for example, carborundum powder), coagulants materials (for example, ferric chloride, sodium hydroxide, chitosan) and/or other chemical agents. The seed coating of the present invention increases the raw weight of a seed by approximately 0.5% to 5% w/w, but can range up to 20% w/w. For example, 0.5%, 1%, 1.5%, 2.3%, 3.7%, 4.5%, 6.7%, 9.5%, 11.4%, 13.6%, 15.9%, 17.2%, 18.8%, 19.4%, or 20% w/w including any integer or fraction thereof, including any hydration added between 0.5% and 20% w/w may be added to seeds during the seed coating treatment method of the present invention. The seed coating non-priming treatment of the present invention is very quick and short in time (from approximately 1 second to approximately 60 minutes, for example, 1.0 second, 2.3 seconds, 3.1 seconds, 6.2 seconds, 11.7 seconds, 59.0 seconds, 84.5 seconds, 118.3 seconds, 169.4 seconds, 214.1 seconds, 240.0 seconds, 5 minutes, 12 minutes, 28 minutes, 46 minutes, or 60 minutes including any integer or fraction thereof), and the coating is external to the seed and does not trigger physiological changes. Seed film coating typically contains a less diluted binder medium than seed coating.

Seed dressing. Seed dressing is the most common method of seed treatment. In seed dressing, a seed is externally covered with a slurry or liquid medium containing a small amount of binder, which is usually 10% or less of the slurry composition, along, in the present invention, with heterologous DNA in the form of naked or virus-based DNA plasmid. Chemical agents, used in agriculture, such as herbicide, pesticides, fungicides, growth hormones, etc., can be added in the seed dressing, which are up-taken by the plant emerging from the seeds, increasing protection or growth vigor. The dressing medium may be composed of a binder diluted in water (binder is less than 10% of the medium), the introduced plasmid (one or more) (in a certain dosage range from 0.1 to 10 μg DNA per seed), color or bio color (0% to 30%), with or without the addition of additives such as, but not limited to, polysaccharides (for example, PEG), abrasive materials (for example, carborundum powder), coagulants materials (for example, ferric chloride, sodium hydroxide, chitosan) and/or other chemical agents. The seed dressing of the present invention increases the raw weight of a seed by approximately 0.5% to 5% w/w, but can range up to 20% w/w. For example, 0.5%, 1%, 1.5%, 2.3%, 3.7%, 4.5%, 6.7%, 9.5%, 11.4%, 13.6%, 15.9%, 17.2%, 18.8%, 19.4%, or 20% w/w including any integer or fraction thereof, including any hydration added between 0.5% and 20% w/w may be added to seeds during the seed dressing treatment method of the present invention. The seed dressing non-priming treatment of the present invention is very quick and short in time (from approximately 1 second to approximately 60 minutes, for example, 1.0 second, 2.3 seconds, 3.1 seconds, 6.2 seconds, 11.7 seconds, 59.0 seconds, 84.5 seconds, 118.3 seconds, 169.4 seconds, 214.1 seconds, 240.0 seconds, 5 minutes, 12 minutes, 28 minutes, 46 minutes, or 60 minutes including any integer or fraction thereof), and the dressing is external to the seed and does not trigger physiological changes.

Seed pelleting. Refers to the process of adding inert materials to seeds to change their size and shape for improved plantability. In addition to adding nucleic acids, chemical agents, used in agriculture, such as herbicide, pesticides, fungicides, growth hormones, etc., can be added in the seed pelleting composition, which are up-taken by the plant emerging from the seeds, increasing protection or growth vigor. The seed pelleting medium may contain heterologous DNA in the form of naked or virus-based DNA plasmid. The seed pelleting of the present invention increases the moisture content of a seed by approximately 0.5% to 5% w/w, but can range up to 20% w/w. For example, 0.5%, 1%, 1.5%, 2.3%, 3.7%, 4.5%, 6.7%, 9.5%, 11.4%, 13.6%, 15.9%, 17.2%, 18.8%, 19.4%, or 20% w/w including any integer or fraction thereof, including any hydration added between 0.5% and 20% w/w may be added to seeds during the seed pelleting treatment method of the present invention. The seed pelleting non-priming treatment of the present invention is very quick and short in time (from approximately 1 second to approximately 60 minutes, for example, 1.0 seconds, 2.3 seconds, 3.1 seconds, 6.2 seconds, 11.7 seconds, 59.0 seconds, 84.5 seconds, 118.3 seconds, 169.4 seconds, 214.1 seconds, 240.0 seconds, 5 minutes, 12 minutes, 28 minutes, 46 minutes, or 60 minutes including any integer or fraction thereof), and the pelleting is external to the seed and does not trigger physiological changes.

Seed viability. Refers to the percent germination potential of a sample of seed.

Stable transformation. Stable DNA introduction is referred to as “stable transformation” resulting in “stably transformed” cell or tissue and refers to the introduction and integration of one or more exogenous polynucleotides into the genome of a cell. The term “stable transformant” refers to a cell which has stably integrated one or more exogenous polynucleotides into the genomic or organellar DNA (chloroplast and/or mitochondria). Plants or parts thereof comprising cell stably transformed with exogenous DNA are typically referred to as “transgenic plants”, “transgenic plant cell” or, in the context of the present invention “transgenic seeds”.

Systemic, symptomless spread. The virus-based DNA construct of a preferred embodiment of the present invention is capable of systemic, symptomless spread in the plant to which it was introduced. As used herein, the term “systemic, symptomless spread” refers to the ability of the plant plasmid vector to spread, for example, from the embryo cell to the developing leaf cells, without inducing the characteristic pathogenic symptoms of the virus.

Transient transformation. Introduction of a heterologous DNA into a cell may be stable or transient. The term “transient” refers to the introduction of one or more exogenous polynucleotides into a cell in the absence of integration of the exogenous polynucleotide into the host cell's genome. This type of DNA introduction may be also referred to as “transient transformation”. The term “transient transformant” thus refers to a cell which has transiently incorporated one or more exogenous polynucleotides. Transiently transformed cells are typically referred to as “non-transgenic” or “non-genetically modified (non-GMO)”.

Treated plants. Refers to plants into which the heterologous DNA has been introduced, regardless if it has been integrated into the plant genome, into its organelles, or remained free in the cytoplasm, or resided as an episome in the nucleus without integration.

True leaves. Any leaves of a plant other than the cotyledons.

Virus based vector. Any plasmid vector that carries any wild type or mutated element or part thereof, which originated from plant viruses, such as promoter, coding sequence or non-coding sequences like coat protein (CP) or an intergenic region (IR). Virus based vectors include vectors that have elements that allow some level of replication and/or spreading.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The agricultural use of genetically-modified plants is a matter of public debate and in many countries is unacceptable by law or regulation. The main considerations voiced against the use of transgenic plants are the fear of inappropriate selection of a transgenic lineage (due to masked deleterious positional effects), possible cross-fertilization with weeds and other crops, further genome alterations due to recombination (especially when copies of endogenous genes are added) and possible transduction of the foreign sequences to plant and soil microorganisms. Introduction of antibiotic-resistant genes to food and the environment is also a major concern.

Bio-safety and environmental aspects can only be concluded upon following actual, carefully controlled, field tests over time. Clearance to conduct such experiments depends on evaluation based on hard laboratory data. One advantage of the methods of the present invention arises from the finding that the TYLCV based constructs appear to be environmentally-friendly and ready for bio-safety-evaluation field tests. Geminiviruses are not seed-transmissible (Kashina et al. 2003. Phytoparasitica 31:188199). The vector forms IL-60, p1470 and pIR are not insect-transmissible even when the plants were colonized with a large number of insect vectors. Thus, the TYLCV based constructs are highly suitable for plant transformation.

In the method of the present invention, the presence of the heterologous DNA in the cell of a treated seed, whether expressed transiently or stably integrated, can be verified by employing any suitable method as is known to a person skilled in the art. Stable transformation of a cell may be detected by isolating genomic DNA and employing Southern blot hybridization with nucleic acid sequences which are capable of binding to one or more of the exogenous polynucleotides or employing polymerase chain reaction with appropriate primers to amplify exogenous polynucleotide sequences. Expression of the transformed DNA can be detected by for example, enzyme-linked immunosorbent assay (ELISA), which detects the presence of a polypeptide encoded by one or more of the exogenous polynucleotides or by detecting the activity of the protein encoded by the exogenous polynucleotide.

Alternatively, in another embodiment of the present invention the exogenous DNA can comprise a marker. A marker provides for the identification and/or selection of a cell, plant, and/or seed expressing the marker. A marker can encode a product, which when expressed at a sufficient level, confers resistance to a selective agent. Such markers and their corresponding selective agents include, but are not limited to, herbicide resistance genes and herbicides; antibiotic resistance genes and antibiotics; and other chemical resistance genes with their corresponding chemical agents. Bacterial drug resistance genes include, but are not limited to, neomycin phosphotransferase II (nptII) which confers resistance to kanamycin, paromycin, neomycin, and G418, and hygromycin phosphotransferase (hph) which confers resistance to hygromycin B. Resistance may also be conferred to herbicides from several groups, including amino acid synthesis inhibitors, photosynthesis inhibitors, lipid inhibitors, growth regulators, cell membrane disrupters, pigment inhibitors, seedling growth inhibitors, including but not limited to imidazolinones, sulfonylureas, triazolopyrimidines, glyphosate, sethoxydim, fenoxaprop, glufosinate, phosphinothricin, triazines, bromoxynil, and the like.

An additional type of marker is a marker the expression of which can be detected by following a biochemical reaction preferably producing color, upon providing of an appropriate substrate. Examples are the GUS (beta-glucuronidase) reporter system, luciderin-luciferase, green-fluorescent protein (GFP) system and the like.

According to certain embodiments, the DNA construct further comprises a regulatory element including, but not limited to, a promoter, an enhancer, and a termination signal.

Among the most commonly used promoters are the nopaline synthase (NOS) promoter (Ebert et al., 1987 Proc. Natl. Acad. Sci. U.S.A. 84:5745-5749), the octapine synthase (OCS) promoter, caulimovirus promoters such as the cauliflower mosaic virus (CaMV) 19S promoter (Lawton et al., 1987 Plant MoI Biol. 9:315-324), the CaMV 35S promoter (Odell et al., 1985 Nature 313:810-812), and the figwort mosaic virus 35S promoter, the light inducible promoter from the small subunit of rubisco, the Adh promoter (Walker et al., 1987 Proc. Natl. Acad. Sci. U.S.A. 84:6624-66280), the sucrose synthase promoter (Yang et al., 1990 Proc. Natl. Acad. Sci. U.S.A. 87:41444148), the R gene complex promoter (Chandler et al., 1989 Plant Cell 1:1175-1183), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al. 1982 Cell 29:1015-1026). A plethora of promoters is described in International Patent Application Publication No. WO 00/18963.

The method of the present invention can unexpectedly be used for introducing any polynucleotide the presence and/or expression of which is of interest, including giving the plant desired property(ies) such as (a) resistance to biotic and abiotic stress conditions, e.g., to insects, nematodes, diseases caused by viral, bacterial fungal and other pathogens pests, resistance to specific herbicide(s) (b) adaptation to hostile conditions such as salt tolerance, drought tolerance, cold and heat tolerance; (c) improved yield quality traits, including but not limited to pigmentation, firmness, type and content of ingredients including sugars, volatiles, oils, fatty acids and/or acids, size, growth rate, dwarf growth habit, time of emergence, time of fruit appearance and ripening; nutritional or commercial value; (d) improved production of edible yields, or of secondary metabolites, for the biopharmaceutical and cosmetic industries either in the form of a protein, secondary metabolites and (e) obtaining a desired growth habit, including determinate, semideterminate and indeterminate (e.g. for tomato), dwarf and normal (e.g. for corn) and plant vigor. The introduced DNA can also confer gene silencing such that the heterologous DNA encodes antisense RNA, siRNA, dsRNA; or additionally, the method can be used for producing raw materials for industries, including but not limited to fiber, wood, oils, resins, etc. as well as other plant traits governed by genetic factors, and/or interaction by genes and environment.

In one embodiment of the present invention, the expressed polynucleotide sequence can encode a molecule which would protect the plant from abiotic stress factors such as drought, heat or chill. Examples include antifreeze polypeptides from Myoxocephalus Scorpius (WO 00/00512) or M. octodecemspinosus, the Arabidopsis thaliana transcription activator CBF1, glutamate dehydrogenases (WO 97/12983, WO 98/11240), calcium-dependent protein kinase genes (WO 98/26045), calcineurins (WO 99/05902), casein kinase from yeast (WO 02/052012), farnesyltransferases (WO 99/06580), ferritin (Deak M. et al. 1999. Nature Biotechnology 17:192-196), oxalate oxidase (WO 99/04013), DREBIA factor (Kasuga M. et al. 1999. Nature Biotech 17:276-286), genes of mannitol or trehalose synthesis such as trehalose-phosphate synthase or trehalose-phosphate phosphatase (WO 97/42326) or by inhibiting genes such as trehalase (WO 97/50561).

The expressed polynucleotide sequence could be a metabolic enzyme for use in the food-and-feed sector. Examples include phytases (GenBank Ace. No.: A 19451) and cellulases.

In another embodiment of the present invention, the expressed polynucleotide sequence can confer resistance to viruses, fungi, insects, nematodes and other pathogens and diseases, by directly attacking the pathogen, turning on the host defenses or by leading to an accumulation of certain metabolites or proteins. Examples include glucosinolates (defense against herbivores), chitinases or glucanases and other enzymes which destroy the cell wall of parasites, ribosome-inactivating proteins (RIPS) and other proteins of the plant resistance and stress reaction as are induced when plants are wounded or attacked by microbes, or chemically, by, for example, salicylic acid, jasmonic acid or ethylene, or lysozymes from non-plant sources such as, for example, T4-lysozyme or lysozyme from a variety of mammals, insecticidal proteins such as Bacillus thuringiensis endotoxin, α-amylase inhibitor or protease inhibitors (cowpea trypsin inhibitor), lectins such as wheatgerm agglutinin, siRNA, antisense RNA, RNAses or ribozymes. Further examples are nucleic acids which encode the Trichoderma harzianum chit42 endochitinase (GenBank Ace. No.: 578423) or the N-hydroxylating, multi-functional cytochrome P-450 (CYP79) protein from Sorghum bicolor (GenBank Ace. No.: U32624), or functional equivalents thereof.

Resistance to pests such as, for example, the rice pest Nilaparvata lugens in rice plants can be achieved by expressing the snowdrop (Galanthus nivalis) lectin agglutinin (Rao et al. 1998. Plant J 15(4):469-77).

The expression of synthetic cry/A(b) and cry/A(c) genes, which encode lepidoptera-specific Bacillus thuringiensis delta-endotoxins can bring about a resistance to insect pests in various plants (Goyal R K. et al. 2000. Crop Protection 19(5):307312).

In another embodiment of the present invention, additional genes which are suitable for pathogen defense comprise “polygalacturonase-inhibiting protein” (PGIP), thaumatine, invertase and antimicrobial peptides such as lactoferrin (Lee T J. et al. 2002. J Amer Soc Horticult Sci 127(2):158164). The expressed polynucleotide sequence can bring about an accumulation of chemicals such as of tocopherols, tocotrienols or carotenoids. One example of such a polynucleotide is phytoene desaturase. Preferred are nucleic acids which encode the Narcissus pseudonarcissus photoene desaturase (GenBank Acc. No.: X78815) or functional equivalents thereof. The expressed polynucleotide sequence can be used for production of nutraceuticals such as, for example, polyunsaturated fatty acids (arachidonic acid, eicosapentaenoic acid or docosahexaenoic acid) by fatty acid elongases and/or desaturases, or for production of proteins with improved nutritional value such as, for example, with a high content of essential amino acids (for example the high-methionine 2S albumin gene of the brazil nut). Preferred are polynucleotide sequences which encode the Bertholletia excelsa high-methionine 2S albumin (GenBank Ace. No.: AB044391), the Physcomitrella patens delta-6-acyl-lipid desaturase (GenBank Ace. No.: AJ222980), the Mortierella alpina delta-6-desaturase (Sakuradani et al. 1999. Gene 238:445-453), the Caenorhabditis elegans delta-5desaturase (Michaelson et al. 1998, FEBS Letters 439:215-218), the Caenorhabditis elegans A5-fatty acid desaturase (des-5) (GenBank Ace. No.: AF078796), the Mortierella alpina delta-5-desaturase (Michaelson et al. JBC 273: 19055-19059), the Caenorhabditis elegans delta-6-elongase (Beaudoin et al. 2000. PNAS 97:6421-6426), the Physcomitrella patens delta-6-elongase (Zank et al. 2000. Biochemical Society Transactions 28:654-657), or functional equivalents of these.

Another preferred construct is the PRN operon. PRN is an antifungal and antibacterial compound produced by certain strains of the bacteria Pseudomonas fluorescens and other bacteria such as Burkholderia cepacia (Chemin et al. (1996) Current Microbiology 32:208-212 and Mozes-Koch et al, (2012) Plant Physiology, Vol. 158, 1883-1892).

In one embodiment of the present invention, the expressed polynucleotide sequence can be used for production of high-quality proteins and enzymes for industrial purposes (for example enzymes, such as lipases) or as pharmaceuticals (such as, for example, antibodies, blood clotting factors, interferons, lymphokine, colony stimulation factor, plasminogen activators, hormones or vaccines, as described, for example, by Hood E E. et al. 1999. Curr Opin Biotechnol 10(4):382-60 and Ma J K. et al. 1999. Curr Top Microbiol Immunol 236:275-92. For example, it has been possible to produce recombinant avidin from chicken albumen and bacterial P-glucuronidase (GUS) on a large scale in transgenic maize plants (Hood et al. 1999. Adv Exp Med Biol 464: 127-47; Review).

In another embodiment of the present invention, the expressed polynucleotide sequence can be also used for obtaining an increased storage ability in cells which normally comprise fewer storage proteins or storage lipids, with the purpose of increasing the yield of these substances for example acetyl-CoA carboxylase. Preferred polynucleotide sequences are those which encode the Medicago sativa acetyl-CoA carboxylase (accase) (GenBank Ace. No.: L25042), or functional equivalents thereof.

Additional examples of expressible polynucleotides include Hepatitis B surface antigen (Kumar G B S et al. 2005. PLANTA 222 (3):484-493), herbicide resistance (Duke, S O. 2005. Pest Management Science 61(21):1-218), interferon (Edelbaum, O. et al. 1992. J. Interferon Res. 12:449-453), T7-RNA polymerase (Zeitoune et al. 1997. Plant Science 141:59-65).

Further examples of polynucleotide sequence which can be expressed in the transformed plants of the present invention are mentioned for example in Dunwell J M. 2000. J Exp Bot. 51:487-96.

The following provide non-limiting examples for applications where reduction of gene expression can be obtained by transforming plant seed with an appropriate heterologous DNA according to teachings of the present invention.

Delayed fruit maturation or a modified maturation phenotype (prolonged maturation, later senescence) can be achieved for example by reducing the gene expression of genes selected from the group consisting of polygalacturonases, pectin esterases, β-1,4)glucanases (cellulases), β-galactanases (β-galactosidases), or genes of ethylene biosynthesis, such as 1-aminocyclopropane-1-carboxylate synthase, adenosylmethionine hydrolase (SAMase), aminocyclopropane-1-carb-oxylate deaminase, aminocyclopropane-1-carboxylate oxidase, genes of carotenoid biosynthesis such as, for example, genes of pre-phytoene biosynthesis or phytoene biosynthesis, for example phytoene desaturases, and O-methyltransferases, acyl carrier protein (ACP), elongation factor, auxin-induced gene, cysteine(thiol) proteinases, starch phosphorylases, pyruvate decarboxylases, chalcone reductases, protein kinases, auxin-related gene, sucrose transporters, meristem pattern gene. Further advantageous genes are described for example in International (PCT) Applications Publication Nos. WO 91/16440, WO 91/05865, WO 91/16426, WO 92/17596. WO 93/07275 or WO 92/04456. Particularly preferred is the reduction of the expression of polygalacturonase for the prevention of cell degradation and mushiness of plants and fruits, for example tomatoes. Nucleic acid sequences such as that of the tomato polygalacturonase gene (GenBank Acc. No.: x14074) or its homologs can preferably used for this purpose.

The reduction of the gene expression of genes encoding storage proteins has numerous advantages, such as, for example, the reduction of the allergenic potential or modification regarding composition or quantity of other metabolites, such as, for example, oil or starch content.

In an embodiment of the present invention, resistance to plant pathogens such as arachnids, fungi, insects, nematodes, protozoans, viruses, bacteria and diseases can be achieved by reducing the gene expression of genes which are essential for the growth, survival, certain developmental stages (for example pupation) or the multiplication of a specific pathogen. Such a reduction can bring about a complete inhibition of the abovementioned steps, or else a delay of same. They can take the form of plant genes which for example make possible the penetration of the pathogen, but may also be homologous pathogen genes. The transformed and expressed heterologous nucleic acid sequence (for example the double-stranded RNA) is directed against genes of the pathogen, such that the pathogen life cycle is interrupted.

In another embodiment of the present invention, virus resistance can be achieved for example by reducing the expression of a viral coat protein, a viral replicase, a viral protease and the like. A large number of plant viruses and suitable target genes are known to the skilled artisan.

In an embodiment of the present invention, resistance to plant pathogens such as, fungi and bacteria, can be achieved by expressing full operon encoding pyrrolnitrin (PRN) (Mozes-Koch et al, (2012) Plant Physiology, Vol. 158, 1883-1892).

In another embodiment of the present invention, reduction of undesired, allergenic or toxic plant constituents such as, for example, glucosinolates or patatin can also be achieved. Suitable target genes are described (in WO 97/16559, inter alia). The target genes which are preferred for reduction of allergenic proteins are described for example by Tada Y et al. (1996) FEBS Lett 391(3):341-345 or Nakamura R (1996) Biosci Biotechnol Biochem 60(8):1215-1221.

Delayed signs of senescence can be achieved by the methods of the present invention. Suitable target genes are, inter alia, cinnamoyl-CoA:NADPH reductases or cinnamoyl-alcohol dehydrogenases. Further target genes are described (in WO 95/07993, inter alia).

Increase of the methionine content by reducing threonine biosynthesis, for example by reducing the expression of threonine synthase (Zeh M et al. 2001. Plant Physiol 127(3): 92-802) can also be achieved by the methods of the present invention.

The heterologous DNA transformed into the plant cell according to the teachings of the present invention can also be employed for the reduction (suppression) of transcription and/or translation of target genes. Thus, the DNA construct can comprises heterologous DNA the expression of which brings about PTGS (post transcriptional gene silencing) or TGS (transcriptional silencing) effects and thus a reduction of the expression of endogenous genes. Such reduction can be achieved for example by expression of an antisense RNA or of a double-stranded RNA, each of which has homology with the endogenous target gene to be suppressed. Also, the expression of a suitable sense RNA can cause a reduction in the expression of endogenous genes, by means of what is known as co-suppression (EP Application Publication No. 0465572). Particularly preferred is the expression of a double-stranded small interfering RNA (siRNA) for reducing the gene expression of a target gene via RNA interference (RNAi). Methods for inhibiting individual target genes using an RNA with double-stranded structure, where the target gene and the region of the RNA duplex have at least partial identity are known to a person skilled in the art.

The present invention provides means and methods for introduction of nucleic acids into plants using non-priming seed treatment. Particularly, the invention combines modified commercial seed treatments (such as coating, film coating, dressing and pelleting) and uploading of heterologous DNA into a plant seed. Surprisingly, the applied heterologous DNA is able to reach seed embryo cells during seed germination. Particularly, the invention provides means and methods of uploading naked and/or viral-based DNA plasmids, which can systemically replicate and spread in the germinated plants without integrating into the plant genome.

According to the present invention, seeds are covered with non-priming treatment medium which includes heterologous DNA plasmids in their naked form. In one embodiment of the present invention, the plasmids are virus based vector DNAs.

In one embodiment of the present invention, the virus based vectors are geminivirus-based expression constructs. The introduced plasmids, which are firmly attached to the seed coat, enter the plantlet cell and reach its nucleus upon germination. There is no need for the use of microorganisms in the method of the present invention.

According to the present invention, the compositions and methods to treat seeds include, but are not limited to, non-priming seed treatment methods such as seed dressing methods, seed coating and film coating methods, and seed pelleting methods.

The non-priming treatment medium of the present invention is composed of water (70% to 99%), the introduced plasmid (one or more) (in a certain dosage range from 0.1 to 10 μg DNA per seed), coating color or bio color (0% to 30%), with or without the addition of additives such as, but not limited to, polysaccharides (for example, PEG), abrasive materials (for example, carborundum powder), coagulants materials (for example, ferric chloride, sodium hydroxide, chitosan) or other chemicals. The non-priming seed treatments of the present invention add between approximately 0.5% to 5% w/w hydration to the seeds; however, as used herein, non-priming seed treatments also include hydration up to 20% w/w. For example, 0.5%, 1%, 1.5%, 2.3%, 3.7%, 4.5%, 6.7%, 9.5%, 11.4%, 13.6%, 15.9%, 17.2%, 18.8%, 19.4%, or 20% w/w hydration including any integer or fraction thereof, including any hydration added between 0.5% and 20% w/w may be added to the seeds during the non-priming treatment method of the present invention. Conversely, in priming methods seeds are hydrated with an addition of approximately 25% to 45% w/w.

The non-priming seed treatment of the present invention is very quick and short in time (from approximately 1 second to approximately 60 minutes, for example, 1.0 seconds, 2.3 seconds, 3.1 seconds, 6.2 seconds, 11.7 seconds, 59.0 seconds, 84.5 seconds, 118.3 seconds, 169.4 seconds, 214.1 seconds, 240.0 seconds, 5 minutes, 12 minutes, 28 minutes, 46 minutes, or 60 minutes, including any integer or fraction thereof), and the coating/dressing/pelleting is external to the seed and does not trigger physiological changes. Conversely, priming seed treatment is a much longer process (from approximately 12 hours to 72 hours or longer) and during priming there is initiation of physiological processes inside the seeds, such as enzymatic activity. In non-priming seed treatments of the present invention, a plasmid will enter into the embryo cells only after sowing the seed in the field and irrigating, whereas in priming treatments, a plasmid will enter into the embryo cells during the priming process itself. In the method of the present invention, seeds are mixed with a non-priming treatment medium and then dried. Seeds can be stored for a period of time or sowed immediately.

### EXAMPLES

The following examples are provided to further illustrate the present invention and are not intended to limit the invention beyond the limitations set forth in the appended claims.

### Example 1

### Coating of Soybean Seeds without Foreign DNA

Soybean seeds were coated with various coating mediums listed below. Batches of 25 soybean seeds (10.5 g each) were mixed by vortexing with 0.3 mL of the coating medium, which is 3% seed weight, for 30 seconds and followed by air drying for 24 hours. The medium was composed of 70% tap water, 30% coating color with or without the addition of 1.5% polyethylene glycol (PEG), 3% PEG, or 1% carborundum.

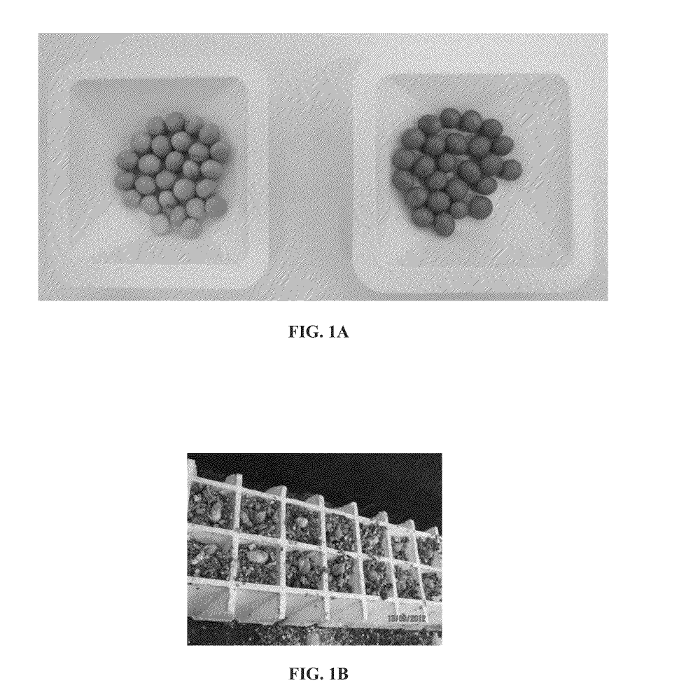

The uniformity of the coating seed treatment was demonstrated by a uniform coverage of the red color on the seed (FIG. 1A, which represents the treatment with 1.5% PEG). The dried treated seeds were sowed on vermiculite and germination rates were recorded after 10 days (FIG. 1B). Untreated dried seeds of the same cultivar, same seed lot were used as control. The results reveal no difference in germination % between control seeds and seeds treated by the methods of the present invention.

### Example 2

### Introducing Plasmid Vectors into Soybean Seeds Using a Coating Treatment

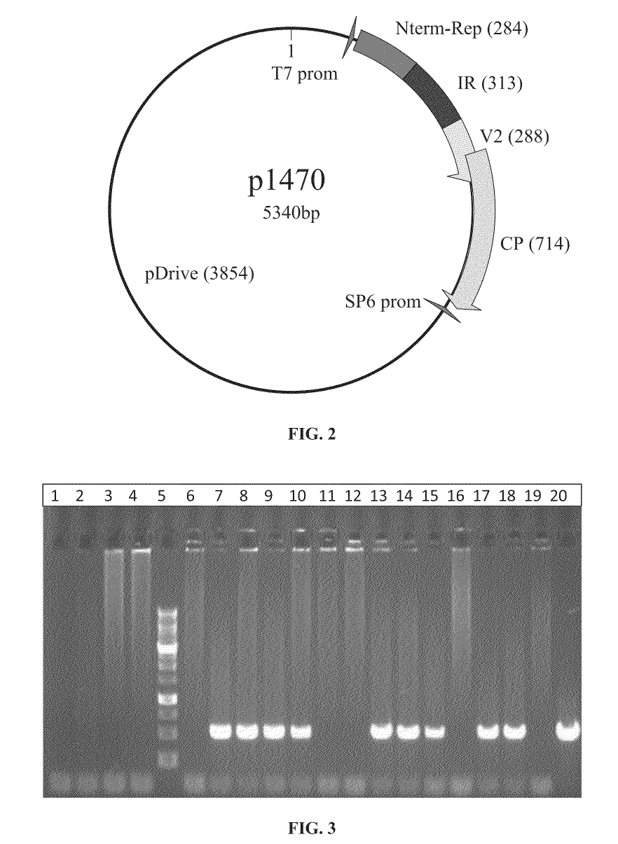

As described in Example 1, soybean seeds were coated with a coating medium composed of 70% tap water, 30% coating colors, and with the addition of 25 μg naked dsDNA plasmid of the viral-based vector p1470 to each batch (containing 25 seeds each). The plasmid construct, described in Patent Application under the name IR-V2-CP (Publication No. WO 2010/004561) is illustrated in FIG. 2. As a control serves seeds that were treated in the same procedure and with the same coating medium but without the addition of the DNA plasmid construct.

The treated seeds were germinated and nested PCR analysis was performed on DNA extracted from true leaves of 14 days old seedlings. The primers used were designed to amplify the fragment of TYLCV coat protein sequence in the p1470 (PCRI with primers A-B followed by PCRII with primers C-D).

Primer A (SEQ ID NO: 1): GCAGTCCGTTGAGGAAACTTACG
Primer B (SEQ ID NO: 2): CATACACTGGATTAGAGGCATGC
Primer C (SEQ ID NO: 3): GTGACTATGTCGAAGCGACCAG
Primer D (SEQ ID NO: 4): GCCTGTTCCTTCATTCCAGAGG

As shown in FIG. 3, positive PCR reaction confirmed the introduction of the plasmid into the plants which emerged from seeds treated by the methods of the present invention.

### Example 3

### Introducing Plasmid Vectors into Soybean Seeds Using Modified Coating Treatments

Soybean seeds were coated with various coating mediums listed in Example 1: and also with 1.5% or 3% PEG, or with 1% carborundum.

The treated seeds were germinated and PCR analysis was performed on DNA extracted from true leaves of 12 days old seedlings. The primers used were the same as in Example 2.

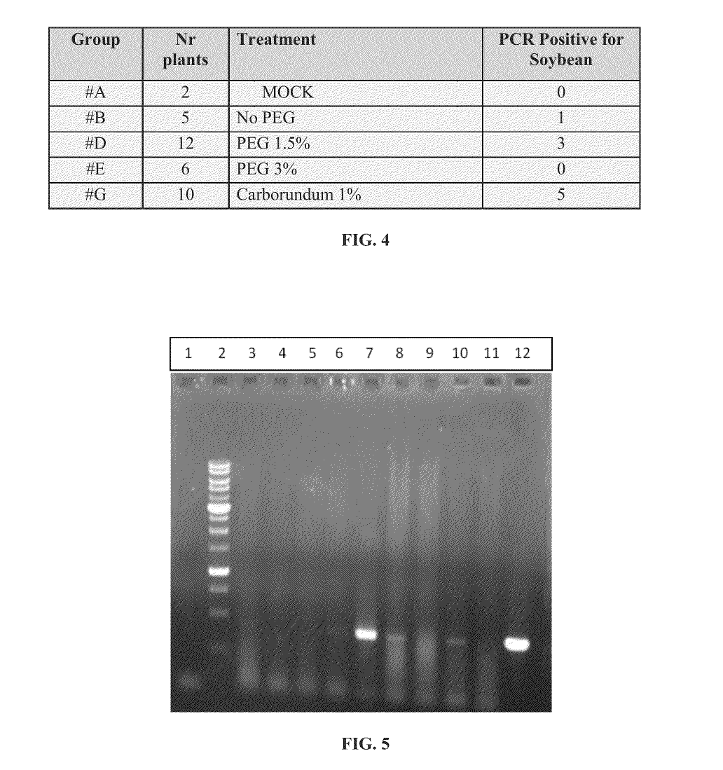

Positive PCR reaction confirmed the introduction of the plasmid into the germinated seeds treated by the methods of the present invention (FIG. 4).

### Example 4

### Introducing Plasmid Vectors into Corn Seeds Using a Dressing Treatment

Two hundred corn seeds (54 g), were mixed with 11 mL of dressing medium, which is 20% seed weight, for 30 seconds and then air dried for 24 hours.

The dressing medium contained 10 mL tap water, 1 mL of coating color, 0.3 g of PEG and 130 μg of plasmid (p1470). The control group was coated with the same medium but without the plasmid.

Dry dressed seeds were planted in vermiculite. After 12 days, the seedlings were sampled for PCR test. The primers E-F used were designed to amplify the fragment of TYLCV coat protein sequence in the p1470.

Primer E (SEQ ID NO: 5): TCACGGTTGCGGTACTGGGCT
Primer F (SEQ ID NO: 6): CCACGCCCGTCTCGAAGGTTC

Positive PCR reaction confirmed the introduction of the plasmid into the germinated seeds treated by the methods of the present invention (FIG. 5).

### Example 5

### Introducing Plasmid Vectors into Canola Seeds Using a Coating Treatment

Two thousand and five hundred seeds of canola (10 g) were coated by mixing by vortexing with a coating medium, which was 6% seed weight, composed of 360 μL water, 214 μL binder, 15 μL PEG-6000, 25 μg naked dsDNA plasmid of the viral-based vector p1470, for 30 seconds and followed by air drying for 24 hours.

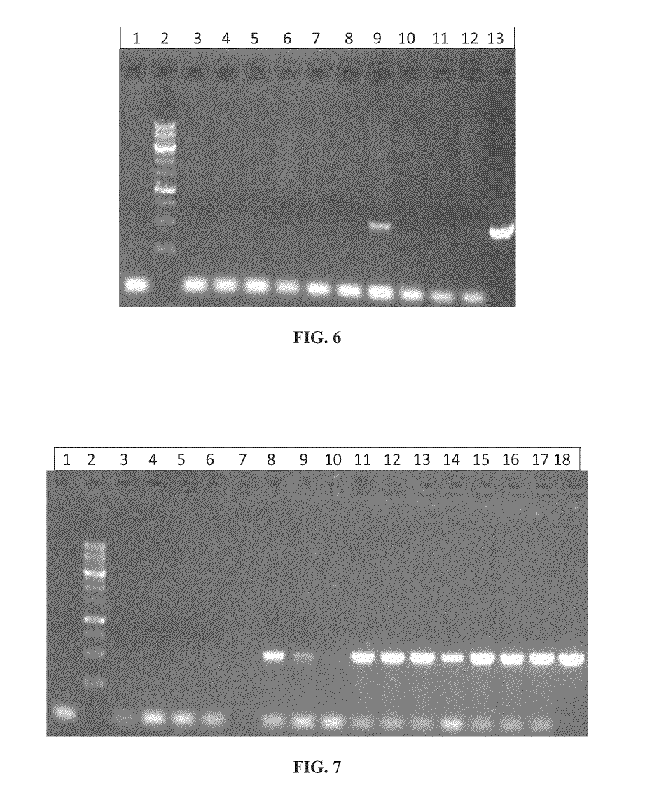

The treated seeds were sown at 1 week after coating and six months after coating and PCR analysis was performed on DNA extracted from true leaves 5 weeks after sowing. The primers used were designed to amplify the fragment of TYLCV coat protein sequence in the p1470 (C-D). As shown in FIG. 6, positive PCR reaction confirmed the introduction of the plasmid into the plants which emerged from seeds treated by the methods of the present invention.

### Example 6

### Introducing Plasmid Vectors into Rice Seeds Using a Coating Treatment

One hundred and fifty seeds of rice (3.5 g) were coated by mixing by vortexing with a 300 μL coating medium, which was 8.5% seed weight, composed of 70% water containing 25 μg naked dsDNA plasmid of the viral-based vector p1470 and 30% binder, for 30 seconds and followed by air drying for 24 hours.

The treated seeds were sown and PCR analysis was performed on DNA extracted from roots one month after sowing. The primers used were designed to amplify the fragment of TYLCV coat protein sequence in the p1470 (C-D). As shown in FIG. 7, positive PCR reaction confirmed the introduction of the plasmid into the plants which emerged from seeds treated by the methods of the present invention.

### Example 7

### Introducing Plasmid Vectors into Rice Seeds Using a Modified Coating Treatment

One hundred and fifty seeds of rice (3.5 g) were coated by mixing by vortexing with a 103 μL coating medium, which was 3% seed weight, composed of 70% water (containing 25 μg naked dsDNA plasmid of the viral-based vector and plasmid carrying GFP gene, driven by 35S promoter) 30% binder, for 30 seconds and followed by air drying for 24 hours.

The treated seeds were sown and GFP expression was monitored by confocal microscopy 2 days after sowing, which showed expression of GFP in the treated rice seed samples; plants have also been successfully checked by PCR (not shown).

### Example 8

### Introducing Plasmid Vectors into Melon Seeds Using a Coating Treatment

Seventy five seeds of melon (2.4 g) were coated by mixing by vortexing with a 2404, coating medium, which was 10% seed weight, composed of 70% water containing 75 μg naked dsDNA plasmid of the viral-based vector p1470 and 30% binder, for 30 seconds and followed by air drying for 24 hours.

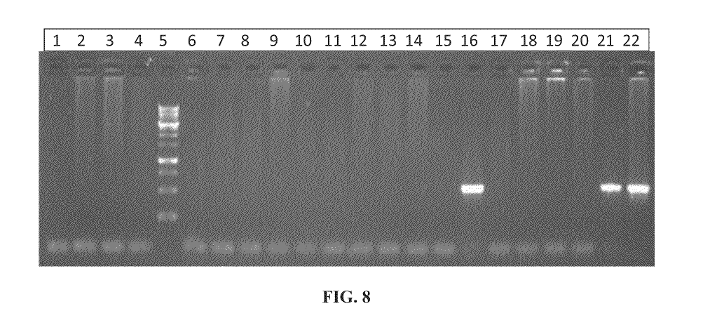

The treated seeds were sown and nested PCR analysis was performed on DNA extracted from true leaves 19 days after sowing. The primers used were designed to amplify the fragment of TYLCV coat protein sequence in the p1470 (PCRI with primers A-B followed by PCRII with primers C-D). As shown in FIG. 8, positive PCR reaction confirmed the introduction of the plasmid into the plants which emerged from seeds treated by the methods of the present invention.

### Example 9

### Introducing Plasmid Vectors into Tomato Seeds Using a Coating Treatment

Fifty seeds of tomato (0.160 g) were coated by mixing by vortexing with a 164, coating medium, which was 10% seed weight, composed of 70% water containing 1.6 μg naked dsDNA plasmid of the viral-based vector p1470 and 1.6 μg naked dsDNA plasmid of the associated “satellite-like” IR-35GUS (GUS driven by 35S promoter), or IR-PRN (PRN operon driven by IR as promoter) 30% binder, for 30 seconds and followed by air drying for 24 hours.

The treated seeds were sown and PCR analysis was performed on DNA extracted from true leaves 3 weeks after sowing. The primers used were designed to amplify the fragment of TYLCV coat protein sequence in the p1470 or sequence of the “satellite like”. Positive PCR reaction confirmed the introduction of both plasmids into the plants which emerged from seeds treated by the methods of the present invention.

### Example 10

### Introducing Plasmid Vectors into Chickpea Seeds Using a Coating Treatment

Thirty seeds of tomato (7.2 g) were coated by mixing by vortexing with a 172.24, coating medium, which was 2.4% seed weight, composed of 70% water containing 30 μg naked dsDNA plasmid of the viral-based vector p1470 and 30 μg naked dsDNA plasmid of the associated “satellite-like” IR-35GUS (GUS driven by 35S promoter), 30% binder, for 30 seconds and followed by air drying for 24 hours.

The treated seeds were sown and real time-PCR analysis was performed on DNA extracted from true leaves 3 weeks after sowing. Positive PCR reaction confirmed the introduction of both plasmids into the plants which emerged from seeds treated by the methods of the present invention.

### Example 11

### Summary of Types of Plant Seeds Treated by the Method of the Present Invention in Relation to their Known Susceptibility to TYLCV Infection

A summary of the types of crops having seed treatment with dsDNA plasmid of the viral-based vector p1470 and its derivatives by dressing, described in the previous examples, or coating in relation to their known susceptibility to TYLCV infection is shown in below in Table 1. Except tomatoes, all species described are known to be not susceptible to TYLCV, particularly monocot plants, such as rice or corn, are not infected by TYLCV. Additionally, TYLCV does not infect plants through seeds. Therefore, the methods of the present invention have generic applicability. An asterisk in Table 1 indicates monocot plants, which are not infected by TYLCV.

TABLE 1

| Crop | TYLCV host? |
|---|---|
| Canola | No |
| Chickpea | No |
| Corn* | No |
| Melon | No |
| Rice* | No |
| Soybean | No |
| Tomato | Yes |

1. A method for introducing nucleic acid into at least one cell of a plant seed embryo comprising contacting a plant seed for less than 60 minutes with a non-priming medium comprising a naked nucleic acid construct or a virus-based DNA construct comprising heterologous DNA, which results in a seed water content increase of less than 20% w/w, thereby obtaining the seed uploaded with the nucleic acid.

2. The method of claim 1, wherein said nucleic acid is present on the surface of the seed coat.

3. The method of claim 1, wherein said non-priming medium is selected from the group consisting of seed coating, film coating, seed dressing and seed pelleting medium.

4. The method of claim 1, wherein the contacting of a plant seed with a non-priming medium results in a seed water content increase of less than 10% w/w.

5. The method of claim 1, wherein the contacting of a plant seed with a non-priming medium results in a seed water content increase of less than 5% w/w.

6. The method of claim 1, wherein the plant seed is in contact with a non-priming medium for less than 5 minutes.

7. The method of claim 1, wherein the virus-based DNA construct comprises viral genes or parts thereof enabling replication and/or symptomless spreading of the construct into adjacent plant cells.

8. The method of claim 7, wherein the virus-based DNA construct is a Geminivirus based construct, and wherein the heterologous DNA construct comprises a polynucleotide sequence encoding a modified Geminivirus coat protein, wherein said modified Geminivirus coat protein comprises a mutation or deletion in nucleotides encoding an N-terminal 100 amino acids.

9. The method of claim 8, wherein the DNA construct further comprises a polynucleotide sequence encoding a modified Geminivirus V2 protein.

10. The method of claim 7, wherein the DNA construct further comprises a bacterial polynucleotide sequence.

11. The method of claim 8, wherein the Geminivirus is Tomato Yellow Leaf Curl Virus selected from the group comprising IL-60, IL-60-BS, p1470 and their derivatives including their transactivatable and inducible pIR satellite like constructs.

12. The method of claim 1, wherein the virus-based DNA construct further comprises a marker for identifying the seed and plants comprising the heterologous DNA.

13. The method of claim 1, wherein the heterologous DNA encodes a product selected from the group consisting of a peptide, a polypeptide, a protein and a RNA molecule.

14. The method of claim 13, wherein the RNA molecule is an inhibitory RNA selected from the group consisting of antisense RNA, dsRNA and siRNA.

15. The method of claim 13, wherein the encoded product confers a desirable agronomic trait selected from the group consisting of resistance to biotic or abiotic stress, increased yield, increased yield quality and preferred growth pattern.

16. The method of claim 1, wherein the heterologous DNA is transiently expressed in at least one cell of the germinated seed embryo and cells derived therefrom.

17. The method of claim 1, wherein the heterologous DNA is incorporated into the genome of the at least one cell of the germinated seed embryo and cells derived therefrom.

18. A seed produced by the method of claim 1, wherein the seed comprises a virus-based DNA construct comprising heterologous DNA.

19. A plant or part thereof produced by growing the seed of claim 18, wherein the plant or part thereof comprises the virus-based DNA construct comprising heterologous DNA.

20. The method of claim 1, wherein the seed is selected from the group consisting of a monocot, dicot and cone-bearing origin.
